(19) United States
(12) Patent Application Publication
(10) Pub. No.: US 2026/0284354 A1
(43) Pub. Date: Sep. 24, 2026

(54) CLINICAL DECISION SUPPORT ALGORITHM FOR FLUID REMOVAL

(71) Applicants: Patrick Hudson Chancy, Franklin Lakes, NJ (US); Brian Longo, Franklin Lakes, NJ (US); Shivapriya Katta, Franklin Lakes, NJ (US); Satya Varaprasad Allumallu, Franklin Lakes, NJ (US); Abhikesh Nag, Franklin Lakes, NJ (US); Becton, Dickinson and Company, Franklin Lakes, NJ (US)

(72) Inventors: Patrick Hudson Chancy, Franklin Lakes, NJ (US); Brian Longo, Franklin Lakes, NJ (US); Shivapriya Katta, Franklin Lakes, NJ (US); Satya Varaprasad Allumallu, Franklin Lakes, NJ (US); Abhikesh Nag, Franklin Lakes, NJ (US)

(21) Appl. No.: 19/471,930

(22) PCT Filed: Apr. 3, 2023
(86) PCT No.: PCT/US2023/017286

§ 371 Date: Oct. 2, 2025

(51) Int. Cl. A61M 25/00; G06N 20/00

(52) U.S. Cl. CPC A61M 25/0017; A61M 2205/18; A61M 2205/502; G06N 20/00

(57) ABSTRACT

In one aspect, there is provided a method that includes receiving, at a processor executing a machine learning model, an input set comprising a measurement of urine output and a time value indicative of time remaining from when the first measurement is performed until a goal time when a target urine output should be reached; predicting, by the machine learning model and using at least the measurement and the time value, an estimated urine output at the goal time; comparing the estimated urine output at the goal time to the target urine output; and generating, based on the comparing of the target urine output and the estimated urine output predicted by the machine learning model, an alert regarding whether the target urine output will be reached by the goal time. Related systems and computer program products are also provided.

### TECHNICAL FIELD

The subject matter described herein relates generally to machine learning to support clinical decision with respect to fluid removal from a patient.

### BACKGROUND

It is often necessary to collect fluids, such as urine, from a patient. To collect urine from the patent urine for example, a urine collection bag may be coupled to a urinary catheter, such as a Foley catheter, that is inserted into the patient's bladder. The patient's urine output may be monitored along with other parameters of the patient. To illustrate further by way of an example, a patient may be given a medicament, such as a diuretic. In this example, a caregiver may administer the diuretic to enable the patient to expel a certain amount of urine. The patient may then be monitored to assess among other things a volume of urine expelled by the patient. If during monitoring, the patient is not expelling an expected amount of urine, the caregiver may reevaluate whether an increase (or decrease) dose of the diuretic should be administered to the patient.

### SUMMARY

Systems, methods, and articles of manufacture, including computer program products, are provided predicting urine output. In one aspect, there is provided a method that includes receiving, at a processor executing a machine learning model, an input set comprising a measurement of urine output and a time value indicative of time remaining from when the first measurement is performed until a goal time when a target urine output should be reached; predicting, by the machine learning model and using at least the measurement and the time value, an estimated urine output at the goal time; comparing the estimated urine output at the goal time to the target urine output; and generating, based on the comparing of the target urine output and the estimated urine output predicted by the machine learning model, an alert regarding whether the target urine output will be reached by the goal time.

In some variations, one or more of the features disclosed herein including the following features can optionally be included in any feasible combination. The receiving further comprises receiving a plurality of input sets corresponding to a plurality of urine outputs and time values until the goal time when the target urine output should be reached. The predicting the estimated urine output is performed after a threshold quantity of input sets are received by the machine learning model. The measurement of urine output indicates a volume of urine or a weight of urine. The urine is collected via a Foley catheter over an interval of time. The input set comprises a total amount of urine output at a measurement time, an average of an hourly urine output at the measurement time, a standard deviation of the hourly urine output at the measurement time, an average hourly urine output during a second half of a urine monitoring session, a previous hour's urine output, an elapsed time since a start of the urine monitoring session, and/or a time remaining until the goal time. The goal time and the target urine output are configured via a user interface and provided to the processor. The machine learning model comprises gradient boosting regression trees machine learning model. The machine learning model is trained, and/or the training is based on at least non-parametric statistical learning. The machine learning model comprises a linear regression machine learning model, a random forest machine learning model, a neural network machine learning model, a gradient boosting regressor machine learning model, and/or a Kalman filter. The alert is provided to a display to present an indication of whether the target urine output will be reached by the goal time. The alert is used to assess whether to change a dosage of a medicament affecting urine output. The comparing is performed by a comparison logic and/or the machine learning model. The machine learning model comprises an ensemble of machine learning models. The ensemble of machine learning models comprises a first machine learning model that predicts a first quantile of a first estimated urine output at the goal time, a second machine learning model that predicts a second quantile of a second estimated urine output at the goal time, and a third machine learning model that predicts a third quantile of a third estimated urine output at the goal time, wherein the first estimated urine output, the second estimated urine output, and the third estimated urine output provide a confidence interval for the estimated urine output at the goal time. The comparing compares the first estimated urine output, the second estimated urine output, and the third estimated urine output to the target urine output. The machine learning model is comprised in a urine collection system, is coupled via a wired or a wireless connection to the urine collection system, is provided as a service to one or more urine collection systems.

Implementations of the current subject matter can include systems and methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the stratification of sepsis patients, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts an example of a plot of dose strength administered to a patient over time, in accordance with some embodiments;

FIG. 2A depicts an example of a urine collection system, in accordance with some embodiments;

FIG. 2B depicts an example of a user interface which may be presented to configure total urine output at a goal time, in accordance with some embodiments;

FIG. 3A depicts a block diagram illustrating an example of a machine learning processor, in accordance with some embodiments;

FIGS. 3B, 3C, and 3D depict examples of user interfaces including alerts regarding whether a patient's urine output will reach a total urine output at a goal time, in accordance with some embodiments;

FIG. 4 depicts a block diagram illustrating an example of an input feature set provided to a machine learning model, in accordance with some embodiments;

FIG. 5 depicts a plot of predicted urine output over time, in accordance with some embodiments;

FIG. 6A depicts a block diagram illustrating an example of training a machine learning model, in accordance with some embodiments;

FIG. 6B depicts a block diagram illustrating an example of an ensemble of machine learning models, in accordance with some embodiments;

FIG. 7 depicts a flowchart illustrating an example of a process for machine learning enabled predictions of urine output, in accordance with some embodiments; and

FIG. 8 depicts a block diagram illustrating an example of a computing system, in accordance with some embodiments.

When practical, similar reference numbers denote similar structures, features, or elements.

### DETAILED DESCRIPTION

A patient administered a diuretic may be monitored to assess among other things an amount of urine expelled by the patient. The amount of urine may be used by a caregiver when assessing whether to increase (or decrease) diuretic dosage for the patient.

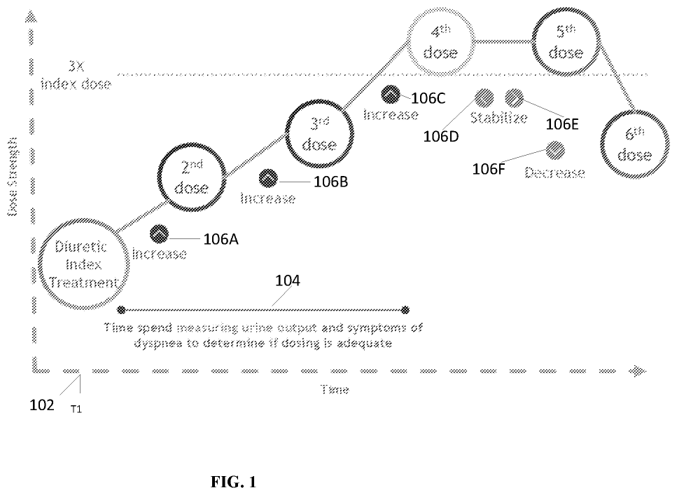

FIG. 1 depicts an example of a plot of dose strength administered to a patient over time. In this example, the patient is administered a diuretic dosage at time T1 102. The patient is monitored over time 104, such that the monitoring includes measuring urine output and/or symptoms of dyspnea, for example. During the monitoring, the caregiver predicts that the patient is not expelling a sufficient amount of urine and as a consequence increases the diuretic dosage at 106A-C. At 106D-E, the caregiver predicts that the patient is expelling a sufficient amount of urine, so there is no change to the diuretic dosages for the 4^{th} and 5^{th} doses. At 106F, the caregiver predicts that the patient has expelled too much urine, so the diuretic dosage is decreased 106F for the 6^{th} dose. FIG. 1 shows by way of an example of how difficult it can be for a caregiver to predict whether a patient is expelling a sufficient amount of urine during a treatment session.

In some embodiments, there is provide a machine learning (ML) model configured to predict whether a patient will reach a target volume of urine output at a goal time. For example, the prediction may be used by a caregiver to determine whether a dosage of a diuretic should be changed (e.g., increased, decreased, or remain the same).

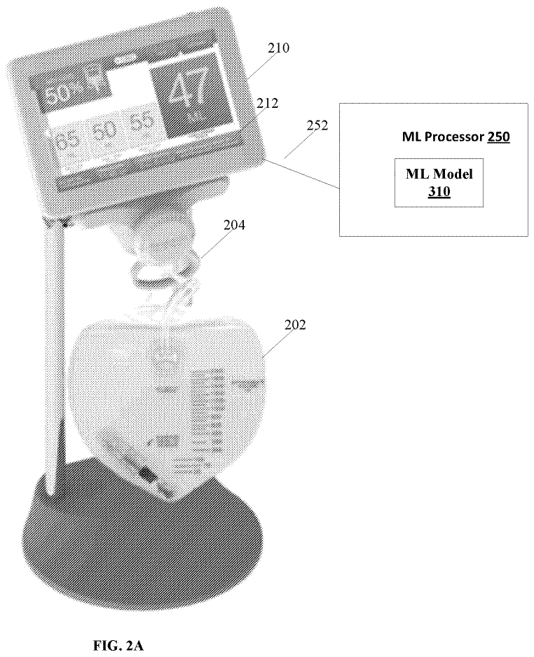

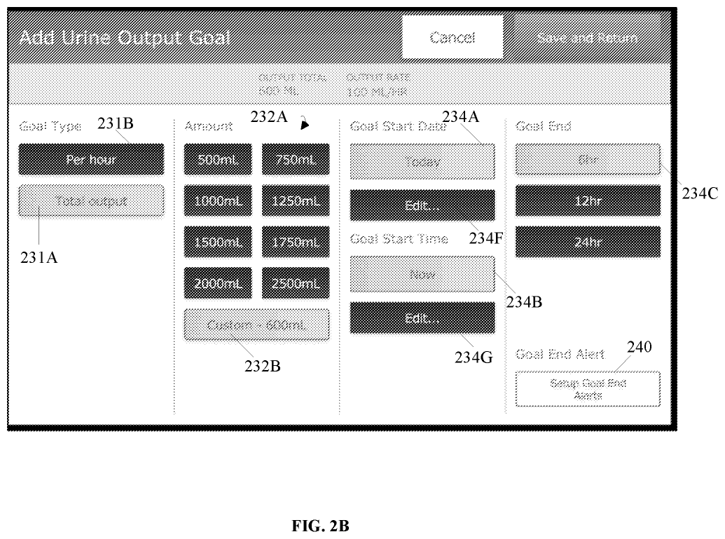

FIG. 2A depicts an example of a urine collection system 200, in accordance with some embodiments. A caregiver may use the urine collection system to collect, measure, and/or monitor urine collected from a patient over time. For example, the urine collection system may include a urine collection bag 202, which can be coupled to a patient's bladder via a catheter, such as a Foley catheter. The urine collection bag may be physically coupled (via, for example, a ring 204) to the urine collection system to enable the volume of the collected urine to be determined over time. As the bag 202 hangs from the ring 204, the weight of the urine in the bag may be measured by the urine collection system from time to time, such as at 15 minute time intervals (or other times as well including 1 minute, 5 minute, 10 minute, 30 minute, 45 minute). The measured weight may then be converted to volume (e.g., volume of urine equals urine weight divided by urine density). The volume of the patient's urine may be stored in at least a database at the urine collection system as part of the patient's electronic medical record (EMR) information and/or may be transmitted (via a wireless link and/or wired link) to a database where the patient's EMR information is stored.

Although some of the examples refer to the amount of collected urine assessed in terms of urine volume, the amount of collected urine may assessed in other terms as well, such as weight, etc.

FIG. 2A further shows a processor 210 including a display 212 (e.g., a touch screen display or other type of display that presents one or more user interfaces to enable interaction with a user). The display may present a user interface depicting the current amount of urine output (e.g., 47 ml for the current hour), past amounts of urine outputs (e.g., 55 ml for the prior hour, 50 ml for 2 hours prior, and 65 ml for 3 hours prior), the percentage fullness of the bag (e.g., 50%), and/or other parameters associated with the urine collection monitoring and/or with the patient being monitored. For example, the diuretic dosage(s), administration time(s) of the dosage(s), urine temperature, patient temperature, total amount of urine to be expelled by the patient by a certain time, and/or other information, alerts, and/or the like.

In operation, the processor 210 may present a user interface 230 as shown at FIG. 2B. The user interface 230 may include user interface elements that can be selected to configure a total amount of urine volume to be expelled by the patient (“target urine volume,” TUV) by a certain goal time (GT). For example, the user interface 230 may include user interface elements 232A corresponding to predefined target urine output volumes, such as 500 ml, 750 ml, 1000 mL, 1250 mL, 1250 mL, 1500 mL, 1750 mL, 2000 mL, 2500 mL, and/or other values of target urine volume. When for example a caregiver selects one of the user interface elements corresponding to the predefined urine output volumes such as 1000 mL, the selected urine output indicates to the processor 210 that the target urine volume (TUV) for the patient being monitored is 1000 mL. Alternatively, or additionally, the user interface 230 may include a user interface element 232B having a customizable TUV. For example, when the user interface element 232B is selected via the touch screen display 212, the selection enables entry of a “custom” TUV that is not listed under the predefined urine output values 232A.

Moreover, the user interface 230 may include user interface elements 234A-C where the goal time (GT) can be configured. In the example of FIG. 2B, the goal time is configured for example to 6 hours 234C, although other times may be configured as well (e.g., 12 hours, 24 hours, or other times as well). Moreover, the configured 6 hour monitoring period can be scheduled to at a given time, such as start now 234B (e.g., today 234A and when now 234B is selected), or at other scheduled times (e.g., when scheduled via a configured start date and time at user interface elements 234F and 234G, respectively). As such, the TUV for the patient being monitored in this example is 600 mL in a GT of 6 hours.

The previous example describes a monitoring session configured to monitor a patient's total output 231A of urine at for example a goal end time of 6 hours 234C. Alternatively, or additionally, a caregiver may select the user interface element 231B to select goal type per (e.g., for each) hour. In other words, the target urine volume (TUV) is for a goal time (GT) of 1 hour, which can repeat for at least one hour. For example, if 100 mL is selected (e.g., as a custom selection at 232B) with a goal type per hour 231B and a goal end time of 6 hours 234C, the target urine output after the 6 hours would be 600 mL. In this example, if the patient's TUV at each hour is below the 100 mL target, an alert may be generated.

Moreover, the user interface 230 may include a user interface element 240, at which alerts may be configured. For example, the alert configuration may include conditions under which an alert should be sent (e.g., patient will not reach the TUV by GT), the types of alerts (e.g., email, audio, haptic, visual, SMS, etc.), and/or recipients of the alerts (e.g., primary caregiver, on duty nurse, etc.).

Referring again to FIG. 2A, the urine collection system 200 may be coupled via a link 252 (e.g., a wired link, a wireless link, a network, the Internet, and/or a bus) to a machine learning (ML) processor 250 including a machine learning model 310, in accordance with some embodiments. The ML processor including the ML model may be configured to predict whether a patient will reach a TUV by a GT (e.g., which were selected at the user interface 230 and provided to the ML processor 250 via the link 252) and output an indication, such as an alert, regarding whether the patient will reach the TUV by the GT.

The ML processor 250 may be comprised in a separate device, such as a processing device including at least one high-speed processor configured to execute ML model(s). Examples of examples of high-speed processors include a Graphic Processing Unit (GPU) chip, an ML (or Artificial Intelligence) chip, and the like. Alternatively, or additionally, the ML processor may be provided as a service (e.g., a cloud service, web service, and/or the like) to one or more urine collection systems. For example, one or more urine collection systems may access (e.g., share) a single ML processor (e.g., via a wired link, a wireless link, a network, the Internet, and/or a bus). Alternatively, or additionally, the ML processor may be comprised in the processor 210 of the urine collection system 200 (e.g., sharing the processing resources with other functions provided by the urine collection system and/or as a separate chip (or chipset) providing the ML resources to the urine collection system).

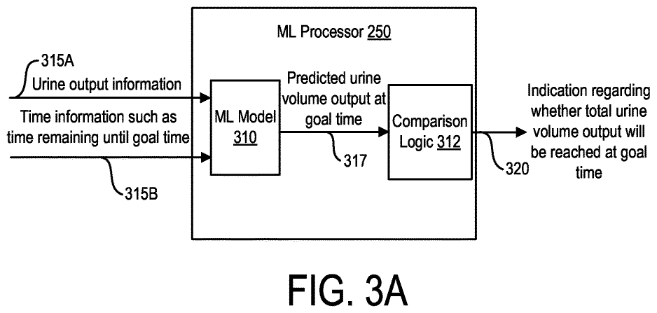

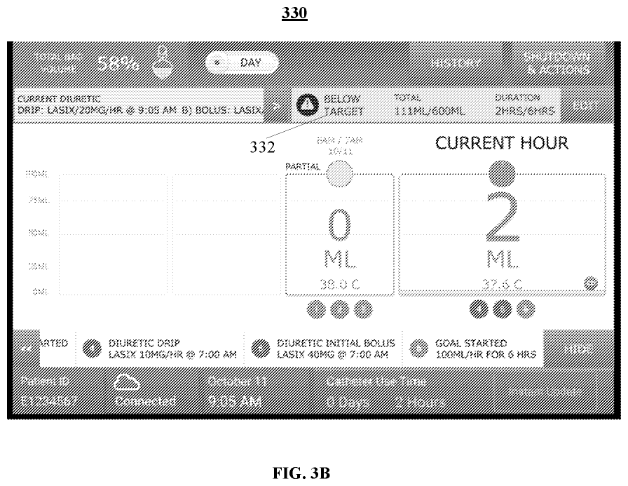

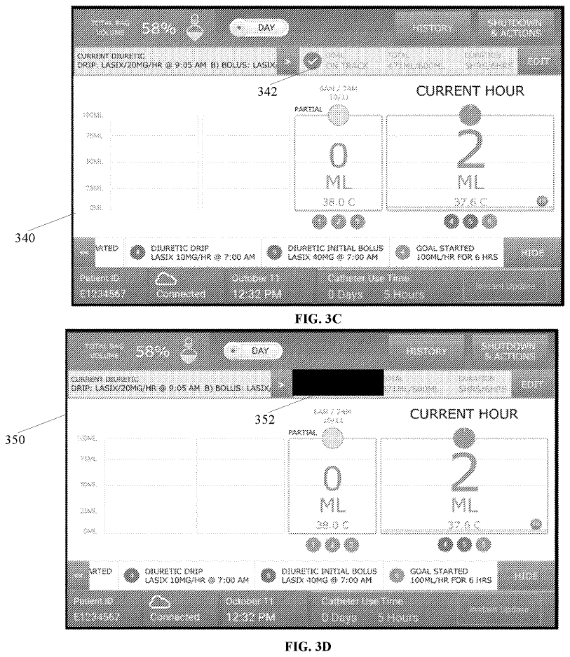

FIG. 3A depicts an example of an implementation of the ML processor 250, in accordance with some embodiments. The ML processor 250 may include the ML model 310 and comparison logic 312. The ML model may be trained (and as such configured) to predict a patient's urine output at one or more times, such as the GT.

In operation, the ML model 310 may receive, as inputs, a patient's urine output information 315A (e.g., measured urine output) and time information 315B (e.g., time remaining until GT). For example, the urine collection system 200 may provide via link 252 to the ML processor 250 a set of inputs 315A-315B. In response to the received inputs 315A-315B, the ML model may provide an output 317 indicating a predicted urine output (e.g., volume or weight of urine) at a certain time, such as a goal time (GT). To illustrate further by way of an example, the urine collection system 200 may provide the inputs 315A-315B comprising one or more measured (e.g., observed) urine output values and one or more corresponding times remaining until the goal time. For example, the inputs 315A-315B may include: a first measurement of 100 mL of total urine volume output and 5 hours remaining from when the first measurement is performed until a goal time, a second measurement of 150 mL of total urine volume output and 4 hours remaining from when the second measurement is performed until the goal time, a third measurement of 250 mL of total urine volume output and 3 hours remaining from when the third measurement is performed until the goal time, and so forth.

To illustrate further by way of an example, the ML model 310 may receive from the urine collection system 200 a urine output value for a current time period (e.g., a second hour of a 6 hour monitoring session) as input 315A and an indication that 4 hours remain from when the urine output measurement is performed until the goal time (GT). In this example, the ML model 310 may generate an output 317 comprising a predicted urine output volume at the GT. The ML processor 250 (e.g., the comparison logic 312) may then compare the output 317 of the predicted urine output volume at GT to a threshold value, such as the TUV which was configured for the GT (e.g., configured via user interface 230 at 232A). Referring to the previous example of 600 mL of TUV at a GT of 6 hours, the ML model may predict a TUV of 111 mL at the 6 hour GT, which is below the threshold 600 mL at 6 hours. In this example, the ML processor (or, e.g., the comparison logic 312) outputs 320 an indication that the predicted urine output (which in this example is 111 mL) will be below configured TUV (which in this example is 600 mL) at the GT (which in this example is 6 hours).

Although FIG. 3A depicts the comparison logic 312 within the ML processor 250, the comparison logic may be included in other processors, such as the processor 210, for example of the urine collection system 200.

FIG. 3B depicts another example of a user interface 330 including a below target user interface element 332 indicating that the predicted urine output volume of the patient will be below the TUV at the GT. Referring to the previous example in which the predicted urine output will be below configured TUV at the GT, the ML processor 250 output 320 may cause or trigger the below target user interface element 332. Moreover, the below target user interface element 332 may trigger other alerts (e.g., haptic, audio, emails, SMS messages) to signal to, for example, a caregiver to reevaluate whether the patient's dosage should be adjusted (which in this example, may suggest an increase in a diuretic). The user interface 330 may be presented at a display, such as the touch screen display 212 at processor 210. Alternatively, or additionally, the user interface 330 (and/or the below target user interface element 332) may be presented at (or provided to) other devices as well.

FIG. 3C depicts another example of a user interface 340 including an on track target user interface element 342 indicating that the predicted urine output volume of the patient is likely to reach the TUV at the GT. For example, the ML processor 250 output 320 may indicate the patient is on track, so the predicted urine output volume of the patient is likely to reach the TUV at the GT. When this is the case, the ML processor 250 output 320 may cause or trigger the on track target user interface element 342. In this example, the caregiver may consider not changing the current dosage of a diuretic. The on track target user interface element 342 may trigger other alerts (e.g., haptic, audio, emails, SMS messages) to notify the caregiver. The user interface 340 may be presented at for example a display, such as the touch screen display 212 at processor 210. Alternatively, or additionally, the user interface 340 (and/or the on track target user interface element 342) may be presented at (or provided to) other devices as well.

FIG. 3D depicts another example of a user interface 350 including above target user interface element 352 indicating that the predicted urine output volume of the patient is over the TUV at the GT. For example, if the predicted urine output volume of the patient is over the TUV at the GT by a threshold amount of urine, the ML processor 250 output 320 may indicate the patient is above target or significantly over the TUV. When this is the case, the ML processor 250 output 320 may cause or trigger the above target user interface element 352. And, the above target user interface element 352 may trigger other alerts (e.g., haptic, audio, emails, SMS messages) to indicate to a caregiver to reevaluate whether the patient's dosage should be adjusted (which in this example, may suggest a decrease in the diuretic). The user interface 350 may be presented at a display, such as the touch screen display 212 at processor 210. Alternatively, or additionally, the user interface 330 (and/or the above target user interface element 352) may be presented at (or provided to) other devices as well.

Although the previous example described an example using only two inputs 315A-B to the ML model 310 to enable the predicted urine output volume 317, other types and/or quantities of inputs may be used as well. In some embodiments, the inputs may include one or more of the following: a total amount of urine output at a measurement (or observation) time, an average hourly urine output at an observation time, a standard deviation of hourly urine output at an observation time, a last (or previous) hour urine output, an elapsed time since start of monitoring by the urine collection system, and a time remaining until GT. These inputs may also be referred to as features, an input set, or an input feature set. Moreover, the inputs may be calculated from measurements of urine output collected over time for a patient (e.g., the average hourly urine output, the standard deviation, and the like).

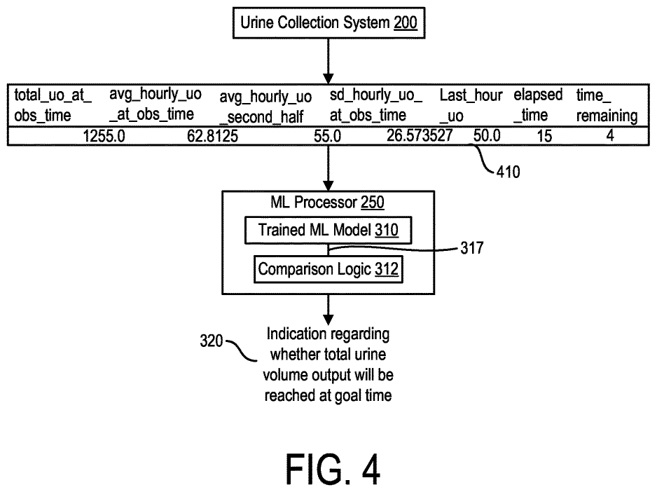

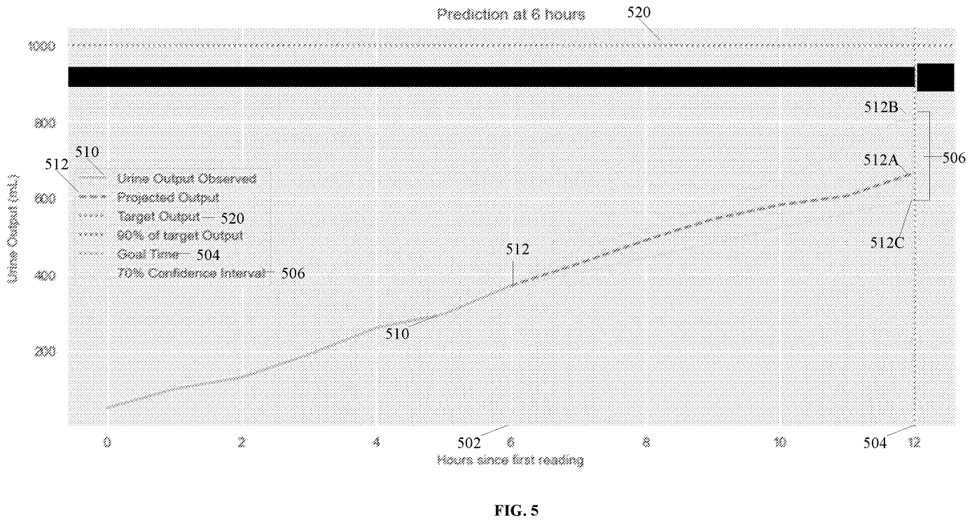

FIG. 4 depicts an example implementation including inputs 410, such as one or more of the following a total amount of urine output at a observation time (total_uo_at_obs_time), an average hourly urine output at an observation time (avg_hourly_uo_at_obs_time), an average hourly urine output during the second half of the patient's session (avg_hourly_uo_second_half), a standard deviation of the hourly urine output at an observation time (sd_hourly_uo_at_obs_time), a last (or previous) hour urine output (last_hour_uo), an elapsed time since start of monitoring by the urine collection system (elapsed_time), and a time remaining until GT (time_remaining). In operation, the inputs 410 may be provided by the urine collection system 200 to the ML processor 250. Alternatively, or additionally, the urine collection system 200 may provide so-called raw data comprising urine observations (or measurements) for a given patient and corresponding time information (e.g., time stamps for when the urine measurements of weight or volume is performed), in which case a processor, such as the ML processor 250 or other processor, may preprocess the raw data to determine and/or provide the inputs 410. In response to the inputs 410, the ML model 310 (which is trained) generates an output 320 indicating whether the urine output will be below, at, or above the configured TUV at the GT.

In the example of FIG. 4, the inputs 410 include information regarding urine output information, such as current urine output (e.g., total amount of urine output at a measurement or observation time such as total_uo_at_obs_time) and past urine output information (e.g., urine output of the prior or last hour, such as last_hour_UO). And, the inputs 410 may include time information, such as elapsed time and time remaining until GT. Moreover, the depicted row of inputs may be referred to as an input set or feature set as these inputs 410 are all associated with the same measurement or observation. If for example another measurement is made, another (or second) row of inputs would be provided as the input or feature set at 410.

In some embodiments, a processor, such as the ML processor 250, the urine collection system 200, and/or other processor, may determine a range or a confidence interval of the predicted urine output volume. The confidence may be configured via a user interface, such as via a selection of user interface element 230 at FIG. 2B. Alternatively, or additionally, the range or confidence interval may be provided as a default value (which may or may not be modified by a caregiver). Furthermore, the comparison logic may, in some embodiments, be incorporated into the ML model 310, in which case the ML model infers the indication 320.

FIG. 5 shows a plot of urine output observed 510 (e.g., solid line representing a plot of actual measurements by the urine collection system 200) and predicted urine outputs 512 (e.g., dashed line of ML model 310 predictions of urine outputs) over time. In this example, the ML model may predict at time 502 the total urine volume output at the goal time 504. The prediction may include a confidence interval (or range) 506. In some embodiments, the confidence interval may be configured via a user interface, such as via a user interface element at user interface 230. Alternatively, or additionally, the confidence interval may be configured as a default. To illustrate further by way of an example, the confidence interval 506 may indicate that there is a 70% chance that the urine output volume prediction at 512A will be within the range 506 defined by a high value of urine output volume 512B and a low value of urine output volume 512C. In some embodiments, the ML model 310 may provide the predicted total urine volume (TUV) output 512A at the goal time (GT) as well as the predictions of the high urine output volume 512B and the low urine output volume 512C, in which case the comparison logic 312 may be used to process 512A-C and determine the indication 320 that the patient reaches the TUV by GT. In the example of FIG. 5, the range 506 (or confidence interval) also indicates that there is a 15% likelihood that the predicted urine output volume will be below the low urine output volume value 512C and a 15% likelihood the predicted urine output volume will be above the high value of the 512B. In the example of FIG. 5, the predicted urine output volume 512A (and even the high urine output volume 512B) is below the TUV 520 for the GT at 504. In this example, an alert (or the output at 320) may be configured to trigger that the patient's urine output volume is below the TUV 520, when the predicted high urine output volume 512B is below the TUV 520. Alternatively, or additionally, an alert (or the output at 320) may be configured to trigger that the patient's urine output volume is below the TUV 520, when the predicted urine output volume 512A is below the TUV 520 but the high predicted high urine output volume 512B is not below the TUV 520. In some embodiments, the confidence interval may be used to determine whether to trigger an alert that the patient will not reach the TUV.

Referring again to FIG. 3A, the ML model 310 may comprise at least one ML model trained and thus configured to predict for a patient whether a patient's urine output will reach a TUV at a GT. In some embodiments, the ML model may comprise Gradient Boosting Regression Trees. In the case of Gradient Boosting Regression Trees (also referred to as Gradient Boosting), the ML model uses a non-parametric statistical learning technique for classification and regression. Non-parametric learning models (e.g., k-nearest neighbors and/or the like) do not rely on specific parameter settings to make predictions. Alternatively, or additionally, the ML model 310 may comprise one or more of the following: a Linear Regression, a Random Forest, neural networks, gradient boosting regressors, and Kalman Filters.

In some implementations, raw patient data may be preprocessed before use in ML model training (as well as ML model inference). For example, urine output (UO) observations measured by the urine collection system 200 (or from other sources or databases of UO observations) may be formatted and/or stored in a database in a tabular form as shown at Table 1 below. In the example, Table 1 shows a “charttime” (which represents a time stamp of when a UO observation is made), a “storetime” indicative of an encounter or a monitoring session with a patient, an “itemid” indicating a corresponding patient, and a urine output (UO) observation in units defined by “valueuom” which in this example is in milliliters (mL).

TABLE 1

| charttime | storetime | itemid | uo | valueuom |
|---|---|---|---|---|
| 2130 Oct. 27 | 2130 Oct. 27 | 226559 | 215.0 | ml |
| 17:03:00 | 17:03:00 | | | |
| 2130 Oct. 27 | 2130 Oct. 27 | 226559 | 350.0 | ml |
| 18:00:00 | 18:06:00 | | | |
| 2130 Oct. 27 | 2130 Oct. 27 | 226559 | 350.0 | ml |
| 19:00:00 | 19:10:00 | | | |
| 2130 Oct. 27 | 2130 Oct. 27 | 226559 | 200.0 | ml |
| 20:00:00 | 21:00:00 | | | |
| 2130 Oct. 27 | 2130 Oct. 27 | 226559 | 200.0 | ml |
| 21:00:00 | 21:58:00 | | | |

In the case of ML model training, the so-called raw data of Table 1 may be preprocessed as noted. For example, the preprocessing may filter the raw data to (1) patients from for example the intensive care unit, (2) patients who have had at least one diuretic administration during their stay, (3) patients who are attached to a Foley catheter, (4) patients who have at least 24 hours of urine output readings, and/or (5) patients who have had at least one urine output reading each hour for the first 24 hours of their stay. This preprocessing is performed to the ML training data to facilitate the training process and to remove patient data for patients that do not have consistent documentation in their urine output readings; in other words, the preprocessing filters data should not be used for training. In an implementation for example, a training set included a training data set of 4155 unique patient stays, although other sized training data sets may be used as well.

Alternatively, or additionally, the processing may also include generating inputs, such as inputs 315A-B or 410 to the ML model 310. As noted, these inputs may also referred to as features. For example, the feature set (or inputs to the ML model) may include one or more of the following inputs (or features):
Total urine output at observation time (total_uo_at_obs_time): The total urine output is the total volume of urine in milliliters that the patient has output from the start of the diuretic therapy to the time a prediction is made;
Average hourly urine output (avg_hourly_uo_at_obs_time): The average hourly urine output is the average urine output per hour since the start of the diuretic therapy;
Average hourly urine output for the second half of stay (avg_hourly_uo_second_half): This represents the average urine output per hour for the second half of the diuretic therapy (e.g., in a 4 hour session, the hourly average urine output for the last two hours);
Standard deviation of the hourly urine output (sd_hourly_uo_at_obs_time): This is the standard deviation of the patient's hourly urine outputs;
Urine output last hour (last_hour_uo): This is the amount of urine output the patient had in a most recent hour;
Elapsed time (elapsed_time): This is the number of hours since the start of the diuretic therapy; and
Time remaining (time_remaining): This is the hours until the goal time.

To illustrate further, the features may be determined from urine observations and time information (e.g., the average and standard deviation of the hourly urine output may be calculated from the so-called raw data of patient(s) urine observations and time information). The use of one or more (if not all) of the noted features may provide better prediction regarding whether a patient's urine output will reach a target urine volume by the goal time, when compared to other types of feature sets. In the case of the average hourly urine output for the second half of stay, this feature may provide a useful indicator of the patient's urine trajectory when compared to the initial measurements when a patient first begins a session for a diuretic and is being monitored by the urine collection system 200. Moreover, in some embodiments, the ML processor 250 may inhibit predictions until after a threshold amount of time or measurements of urine output to help ensure accurate predictions. For example, the ML processor may be configured with a threshold amount of measurements (e.g., 4 urine output measurements over a 1 hour period) before enabling the predictions 317 and output indications 320 regarding TUV at GT.

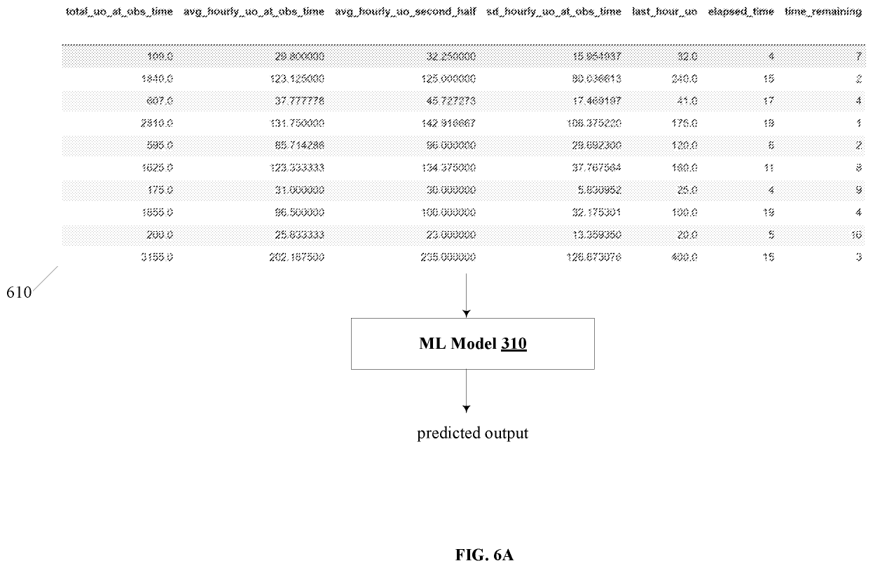

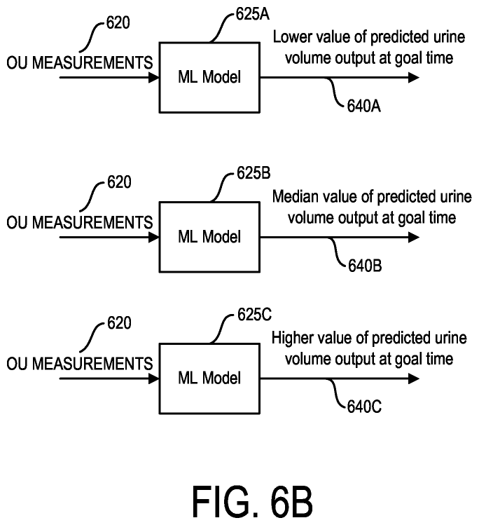

After the features are determined and/or calculated, the training data 610 as shown at FIG. 6A may be provided to the ML model 310 to train the ML model. In this example, each row of the training data represents a patient stay (e.g., ICU patient stay) with a corresponding column for the features noted above. In the case of supervised or semi-supervised learning of the ML model, the training data may include a label at each of the rows indicating the predicted outcome (e.g., predicted urine output at goal time and/or indication regarding whether total urine volume output will be reached at GT). In the case of supervised or semi-supervised learning of the ML model, the ML model adjusts it configuration (e.g., weights) until it learns (e.g., converges) to generate the predicted outcome given the input features. After training of the ML model 310 at FIG. 6A, the ML model may be used to perform inferences using new input data (e.g., 315A-B/410) from a patient's urine observation information (e.g., perform the predictions 317 and output indications 320 regarding TUV at GT).

In some embodiments, the ML model 310 may comprise an ensemble of a plurality of ML models. For example, the ML model 310 may comprise three ML models. For example, a first ML model is trained to provide a lower urine output volume prediction, such as 512C. The second ML model is trained to provide a median urine output volume prediction (e.g., 512A), and the third ML model is trained to provide a high urine output volume prediction, such as 512B. In this way, the ensemble can provide a range 506 (e.g., confidence interval) of predicted urine output volumes rather than a single point prediction of urine output volume. Rather than predict a single urine output volume (e.g., urine output volume 512A) for example, the ensemble of ML models provides a predicted confidence interval or range 506. In some embodiments, the three ML models comprise three Gradient Boosting Regression Trees models or three Random Forest models, although other types of ML models may be used as well.

In some embodiments, the first ML model may be trained to provide a prediction for a lower percentile mark for the expected urine output volume. For example, the first model may provide the lower urine output volume prediction 512C, which may correspond to the lower percentile of the range, such as the lower 10%, 15%, 20%, 25%, or other value of the lower interval. With the lower percentile at 20%, roughly 80% of the predictions made by ML model will be above the lower interval prediction. Similarly, the third ML model may provide the high urine output volume prediction 512B, which may correspond to the higher percentile of the range, such as the lower 90%, 85%, 80%, 75% or other value of the higher interval. With the high percentile at 80%, 80% of the urine output volume predictions will fall below the upper interval mark. With this configuration of the confidence interval, if the ML processor predicts that a patient will not meet a total urine volume output by a goal time, it will be correct 80% of the time in this example. FIG. 6B depicts an example implementation of the ensemble ML models 625A-C, each of which provides the low prediction 640A, median prediction 640B, and high prediction 640C. The outputs 640A-640C may be coupled to comparison logic (e.g., comparison logic 312) which compares the outputs 640A-C to the TUV and outputs (e.g., output 320) an indication regarding whether the TUV output will be reached at GT. Although FIG. 6B shows an ensemble of three ML models, other quantities of ML models may be used in the ensemble.

As noted, the ML model 310 (or ensemble) may be trained (e.g., in a training phase) using raw data filtered as noted above from a large set of patients. For example, the ML model 310 may be initially trained using data from a large, general database of patient data and filtered as noted above with respect to (1)-(5). Alternatively, or additionally, the ML model training data may include data obtained from observations provided by one or more urine collection systems 200.

During the training phase of the ML model 310, the ML model(s) may consume training data, which may be a relatively large amount of data. The training data may be characterized as labeled data indicating “known” information with respect to the training data. During training, the ML model learns (configures itself) to predict the output (which is known or labeled) based on the input. Specifically, the ML model learns how to predict urine output level by ingesting training data. This training data may be similar to the training data 610 depicted at FIG. 6A from a plurality of patients, where the urine output (UO) in milliliters is known. In the case of the ensemble ML models noted above, the lower, median, and upper models are trained by minimizing the a-quantile regression function:

$$l ( \vec{y} , F ( \vec{x} ) ) = {\sum }_{i = 0}^{num \_ rows} max ( ( 1 - α ) ( {y}_{i} - F ( {x}_{i} ) ) , α ( F ( {x}_{i} ) - {y}_{i} ) )$$

where α is a quantile (e.g., 0.2, 0.5, and 0.8 respectively, although the quantiles may take other values as well), yᵢ is the i^{th} output of the ML models, F(xᵢ) is the predicted i^{th} output, and xᵢ is the input data for the i^{th} observation.

When the ML model(s) are trained, the ML model(s) may, as noted, then be used to predict (in the inference phase) urine output at a goal time (GT) for at least one patient. For example, the trained ML model(s) may for at least one patient (whose urine output measurement data was not part of the training data as it is so-called “new” data) predict the urine output volume for the GT as noted above with respect to FIGS. 2A, 3A, and 4.

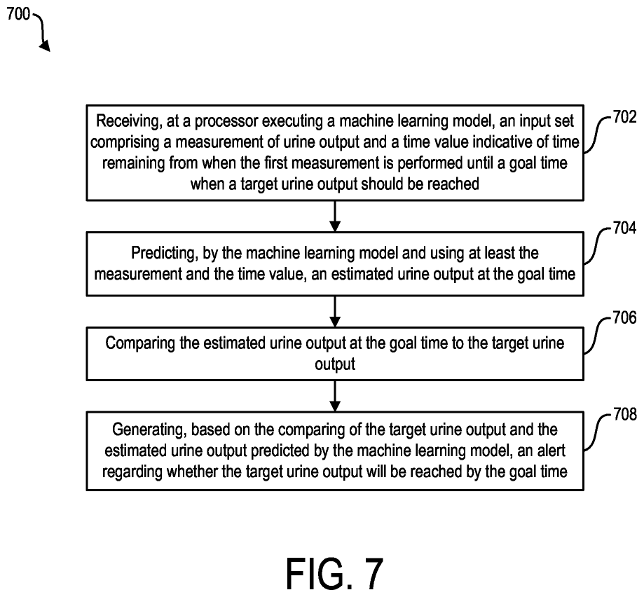

FIG. 7 depicts a flowchart illustrating an example of a process 700 for using ML to predict whether a patient's urine output volume will reach a target urine output (TUV) a goal time (GT).

At 702, the process may include receiving, at a processor executing a machine learning model, an input set comprising a measurement of urine output and a time value indicative of time remaining from when the first measurement is performed until a goal time when a target urine output should be reached. For example, the ML processor 250 including the ML model 310 (which has been trained with training data to predict urine output at a goal time) may receive at least a first measurement of urine output over a first time interval. For example, the input set may correspond to total urine output at observation time (see, e.g., total_uo_at_obs_time as noted above) or some other value indicative of a patient's observation/measurement of urine collected via a Foley catheter and measured by the urine collection system 200. Moreover, the time value indicative of time remaining until a goal time (GT) when a target urine output should be reached may correspond to a time remaining until the goal time is reached (see, e.g., time_remaining). Referring to the example of FIG. 4, the measurement may correspond to 1255.0 ML and the time value may correspond to 4 hours. Although this example refers to two inputs, the ML model may receive other values as part of the input feature set as well, such as an average of an hourly urine output at the measurement time, a standard deviation of the hourly urine output at the measurement time, an average hourly urine output during a second half of a monitoring session of urine output, a previous hour's urine output, an elapsed time since a start of the monitoring session of urine output, and/or the like.

At 704, the process may include predicting, by the machine learning model and using at least the measurement and the time value, an estimated urine output at the goal time, in accordance with some embodiments. For example, the ML model 310 may generate a prediction of the estimated urine output as noted above with respect to 317 at FIGS. 3A and 4.

At 706, the process may include comparing the estimated urine output at the goal time to the target urine output. For example, the estimated urine output 317 (which is predicted by the ML model 310) may be compared to the target urine volume. This comparison may be performed by the comparison logic 312. Alternatively, or additionally, the comparison may be performed by the ML model.

At 708, the process may include generating, based on a comparison of the target urine output and the estimated urine output predicted by the trained machine learning model, an alert regarding whether the target urine output will be reached by the goal time. For example, the estimated urine output 317 (which is predicted by the ML model 310) may be compared to the target urine output (which may be configured via a user interface, such as user interface 230, for the patient). In the example of FIGS. 3A and 4, the comparison logic 312 performs this comparison. If the comparison indicates that the estimated urine output is less than the target urine output at goal time for example, an alert (which indicates that the target urine output at goal time will not be reached) is generated. An example of the below target alert is depicted at 332 at FIG. 3B. If the comparison indicates that the estimated urine output is more than the target urine output at goal time for example, an alert (which indicated that the target urine output at goal time will be exceeded) is generated. An example of the above target alert is depicted at 352 at FIG. 3D. And, if the comparison indicates that the estimated urine output will reach the target urine output at goal time for example, an alert (which indicates that the target urine output is on track) will be generated. An example of the on track alert is depicted at 342 at FIG. 3C. Alternatively, or additionally, the alert generation may be performed by the ML model.

In some embodiments, the ML processor 250 may generate the indication 320 after a threshold quantity of input information 315A-B (and/or 410) is received and processed by the ML model 310. For example, the ML processor 250 may require a minimum quantity of a patient's urine output measurements before making predictions at 317 and/or 320.

In some embodiments, the receiving at 702 further includes receiving a plurality of input sets corresponding to a plurality of urine outputs, each of which includes a corresponding time value until the goal time when the target urine output should be reached. As noted in the example of FIG. 4, the input row (at 410 or portion thereof) may comprise a first input set while another measurement/observation may provide a second input set. For example, the first input set may include a total amount of urine output at a measurement time, an average of an hourly urine output at the measurement time, a standard deviation of the hourly urine output at the measurement time, an average hourly urine output during a second half of a urine monitoring session, a previous hour's urine output, an elapsed time since a start of the urine monitoring session, and/or a time remaining until the goal time, while the second input set may correspond to these values for another measurement/observation of the patient.

In some embodiments, the predicting the estimated urine output is performed after a threshold quantity of input sets are received by the machine learning model. As noted, the ML model may be inhibiting its predictions until a threshold quantity of inputs are provided.

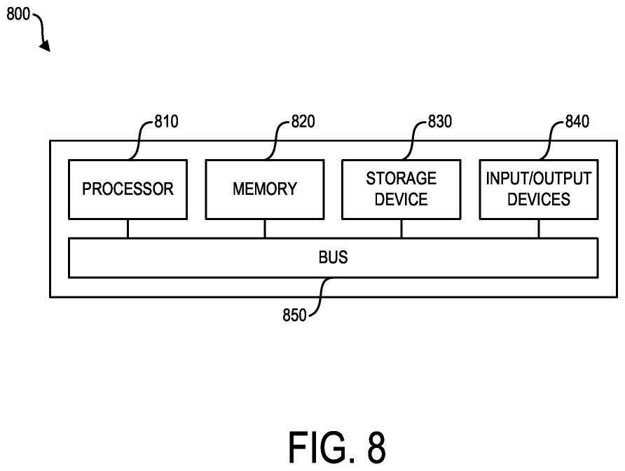

FIG. 8 depicts a block diagram illustrating an example of computing system 800, in accordance with some embodiments. The computing system 800 may be used (at least in part) to provide aspects of one or more of the following: processor 210, ML processor 250, the ML model 310, and the comparison logic 312.

As shown in FIG. 8, the computing system 800 can include a processor 810, a memory 820, a storage device 830, and input/output devices 840. The processor 810, the memory 820, the storage device 830, and the input/output devices 840 can be interconnected via a system bus 850. The processor 810 is capable of processing instructions for execution within the computing system 800. Such executed instructions can implement one or more components of, for example, the patient stratification system 100 and/or the like. In some implementations of the current subject matter, the processor 810 can be a single-threaded processor. Alternately, the processor 810 can be a multi-threaded processor. The process may be a multi-core processor have a plurality or processors or a single core processor. The processor 810 is capable of processing instructions stored in the memory 820 and/or on the storage device 830 to display graphical information for a user interface provided via the input/output device 840. The memory 820 is a computer readable medium such as volatile or non-volatile that stores information within the computing system 800. The memory 820 can store data structures representing configuration object databases, for example. The storage device 830 is capable of providing persistent storage for the computing system 800. The storage device 830 can be a floppy disk device, a hard disk device, an optical disk device, a tape device, or other suitable persistent storage means. The input/output device 840 provides input/output operations for the computing system 800. In some implementations of the current subject matter, the input/output device 840 includes a keyboard and/or pointing device. In various implementations, the input/output device 840 includes a display unit for displaying graphical user interfaces. According to some implementations of the current subject matter, the input/output device 840 can provide input/output operations for a network device. For example, the input/output device 840 can include Ethernet ports or other networking ports to communicate with one or more wired and/or wireless networks (e.g., a local area network (LAN), a wide area network (WAN), the Internet).

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Example 1. A method comprising: receiving, at a processor executing a machine learning model, an input set comprising a measurement of urine output and a time value indicative of time remaining from when the first measurement is performed until a goal time when a target urine output should be reached; predicting, by the machine learning model and using at least the measurement and the time value, an estimated urine output at the goal time; comparing the estimated urine output at the goal time to the target urine output; and generating, based on the comparing of the target urine output and the estimated urine output predicted by the machine learning model, an alert regarding whether the target urine output will be reached by the goal time.

Example 2. The method of Example 1, wherein the receiving further comprises receiving a plurality of input sets corresponding to a plurality of urine outputs and time values until goal time when the target urine output should be reached.

Example 3. The method of any of Examples 1-2, wherein the predicting the estimated urine output is performed after a threshold quantity of input sets are received by the machine learning model.

Example 4. The method of any of Examples 1-3, wherein the measurement of urine output indicates a volume of urine or a weight of urine, wherein the urine is collected via a Foley catheter over an interval of time.

Example 5. The method of any of Examples 1-4, wherein the input set comprises a total amount of urine output at a measurement time, an average of an hourly urine output at the measurement time, a standard deviation of the hourly urine output at the measurement time, an average hourly urine output during a second half of a urine monitoring session, a previous hour's urine output, an elapsed time since a start of the urine monitoring session, and/or a time remaining until the goal time.

Example 6. The method of any of Examples 1-5, wherein the goal time and the target urine output are configured via a user interface and provided to the processor.

Example 7. The method of any of Examples 1-6, wherein the machine learning model comprises gradient boosting regression trees machine learning model.

Example 8. The method of any of Examples 1-7, wherein the machine learning model is trained, and/or wherein the training is based on at least non-parametric statistical learning.

Example 9. The method of any of Examples 1-8, wherein the machine learning model comprises a linear regression machine learning model, a random forest machine learning model, a neural network machine learning model, a gradient boosting regressor machine learning model, and/or a Kalman filter.

Example 10. The method of any of Examples 1-9 further comprising: providing the alert to a display to present an indication of whether the target urine output will be reached by the goal time.

Example 11. The method of any of Examples 1-10, wherein the alert is used to assess whether to change a dosage of a medicament affecting urine output.

Example 12. The method of any of Examples 1-11, wherein the comparing is performed by a comparison logic and/or the machine learning model.

Example 13. The method of any of Examples 1-12, wherein the machine learning model comprises an ensemble of machine learning models.

Example 14. The method of any of Examples 1-13, wherein the ensemble of machine learning models comprises a first machine learning model that predicts a first quantile of a first estimated urine output at the goal time, a second machine learning model that predicts a second quantile of a second estimated urine output at the goal time, and a third machine learning model that predicts a third quantile of a third estimated urine output at the goal time, wherein the first estimated urine output, the second estimated urine output, and the third estimated urine output provide a confidence interval for the estimated urine output at the goal time.

Example 15. The method of any of Examples 1-14, wherein the comparing compares the first estimated urine output, the second estimated urine output, and the third estimated urine output to the target urine output.

Example 16. The method of any of Examples 1-5, wherein the machine learning model is comprised in a urine collection system, is coupled via a wired or a wireless connection to the urine collection system, is provided as a service to one or more urine collection systems.

Example 17. An apparatus comprising: at least one processor; and
at least one memory including code which when executed by the at least one processor causes operations comprising: receiving, at a machine learning model, an input set comprising a measurement of urine output and a time value indicative of time remaining from when the first measurement is performed until a goal time when a target urine output should be reached; predicting, by the machine learning model and using at least the measurement and the time value, an estimated urine output at the goal time; comparing the estimated urine output at the goal time to the target urine output; and generating, based on the comparing of the target urine output and the estimated urine output predicted by the machine learning model, an alert regarding whether the target urine output will be reached by the goal time.

Example 18. The apparatus of Example 17, wherein the receiving further comprises receiving a plurality of input sets corresponding to a plurality of urine outputs and time values until goal time when the target urine output should be reached.

Example 19. The apparatus of any of Examples 17-18, wherein the predicting the estimated urine output is performed after a threshold quantity of input sets are received by the machine learning model.

Example 20. The apparatus of any of Examples 17-19, wherein the measurement of urine output indicates a volume of urine or a weight of urine, wherein the urine is collected via a Foley catheter over an interval of time.

Example 21. The apparatus of any of Examples 17-20, wherein the input set comprises a total amount of urine output at a measurement time, an average of an hourly urine output at the measurement time, a standard deviation of the hourly urine output at the measurement time, an average hourly urine output during a second half of a urine monitoring session, a previous hour's urine output, an elapsed time since a start of the urine monitoring session, and/or a time remaining until the goal time.

Example 22. The apparatus of any of Examples 17-21, wherein the goal time and the target urine output are configured via a user interface and provided to the apparatus.

Example 23. The apparatus of any of Examples 17-22, wherein the machine learning model comprises gradient boosting regression trees machine learning model.

Example 24. The apparatus of any of Examples 17-23, wherein the machine learning model is trained, and/or wherein the training is based on at least non-parametric statistical learning.

Example 25. The apparatus of any of Examples 17-24, wherein the machine learning model comprises a linear regression machine learning model, a random forest machine learning model, a neural network machine learning model, a gradient boosting regressor machine learning model, and/or a Kalman filter.

Example 26. The apparatus of any of Examples 17-25 further comprising providing the alert to a display to present an indication of whether the target urine output will be reached by the goal time.

Example 27. The apparatus of any of Examples 17-26, wherein the alert is used to assess whether to change a dosage of a medicament affecting urine output.

Example 28. The apparatus of any of Examples 17-27, wherein the comparing is performed by a comparison logic and/or the machine learning model.

Example 29. The apparatus of any of Examples 17-28, wherein the machine learning model comprises an ensemble of machine learning models.

Example 30. The apparatus of any of Examples 17-29, wherein the ensemble of machine learning models comprises a first machine learning model that predicts a first quantile of a first estimated urine output at the goal time, a second machine learning model that predicts a second quantile of a second estimated urine output at the goal time, and a third machine learning model that predicts a third quantile of a third estimated urine output at the goal time, wherein the first estimated urine output, the second estimated urine output, and the third estimated urine output provide a confidence interval for the estimated urine output at the goal time.

Example 31. The apparatus of any of Examples 17-30, wherein the comparing compares the first estimated urine output, the second estimated urine output, and the third estimated urine output to the target urine output.

Example 32. The apparatus of any of Examples 17-31, wherein the machine learning model is comprised in a urine collection system, is coupled via a wired or a wireless connection to the urine collection system, is provided as a service to one or more urine collection systems.

Without in any way limiting the scope, interpretation, or application of the claims appearing below, a technical effect of one or more of the example embodiments disclosed herein may include enhanced prediction of whether a target urine output amount will be reached by a goal time.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

What is claimed:

1. A method comprising:
receiving, at a processor executing a machine learning model, an input set comprising a measurement of urine output and a time value indicative of time remaining from when the first measurement is performed until a goal time when a target urine output should be reached;
predicting, by the machine learning model and using at least the measurement and the time value, an estimated urine output at the goal time;
comparing the estimated urine output at the goal time to the target urine output; and
generating, based on the comparing of the target urine output and the estimated urine output predicted by the machine learning model, an alert regarding whether the target urine output will be reached by the goal time.

2. The method of claim 1, wherein the receiving further comprises receiving a plurality of input sets corresponding to a plurality of urine outputs and time values until goal time when the target urine output should be reached.

3. The method of claim 2, wherein the predicting the estimated urine output is performed after a threshold quantity of input sets are received by the machine learning model.

4. The method of claim 1, wherein the measurement of urine output indicates a volume of urine or a weight of urine, wherein the urine is collected via a Foley catheter over an interval of time.

5. The method of claim 1, wherein the input set comprises a total amount of urine output at a measurement time, an average of an hourly urine output at the measurement time, a standard deviation of the hourly urine output at the measurement time, an average hourly urine output during a second half of a urine monitoring session, a previous hour's urine output, an elapsed time since a start of the urine monitoring session, and/or a time remaining until the goal time.

6. The method of claim 1, wherein the goal time and the target urine output are configured via a user interface and provided to the processor.

7. The method of claim 1, wherein the machine learning model comprises gradient boosting regression trees machine learning model.

8. The method of claim 1, wherein the machine learning model is trained, and/or wherein the training is based on at least non-parametric statistical learning.

9. The method of claim 1, wherein the machine learning model comprises a linear regression machine learning model, a random forest machine learning model, a neural network machine learning model, a gradient boosting regressor machine learning model, and/or a Kalman filter.

10. The method of claim 1 further comprising:
providing the alert to a display to present an indication of whether the target urine output will be reached by the goal time.

11. The method of claim 1, wherein the alert is used to assess whether to change a dosage of a medicament affecting urine output.

12. The method of claim 1, wherein the comparing is performed by a comparison logic and/or the machine learning model.

13. The method of claim 1, wherein the machine learning model comprises an ensemble of machine learning models.

14. The method of claim 13, wherein the ensemble of machine learning models comprises a first machine learning model that predicts a first quantile of a first estimated urine output at the goal time, a second machine learning model that predicts a second quantile of a second estimated urine output at the goal time, and a third machine learning model that predicts a third quantile of a third estimated urine output at the goal time, wherein the first estimated urine output, the second estimated urine output, and the third estimated urine output provide a confidence interval for the estimated urine output at the goal time.

15. The method of claim 14, wherein the comparing compares the first estimated urine output, the second estimated urine output, and the third estimated urine output to the target urine output.

16. The method of claim 1, wherein the machine learning model is comprised in a urine collection system, is coupled via a wired or a wireless connection to the urine collection system, is provided as a service to one or more urine collection systems.

17. An apparatus comprising:
at least one processor; and
at least one memory including code which when executed by the at least one processor causes operations comprising:
receiving, at a machine learning model, an input set comprising a measurement of urine output and a time value indicative of time remaining from when the first measurement is performed until a goal time when a target urine output should be reached;
predicting, by the machine learning model and using at least the measurement and the time value, an estimated urine output at the goal time;
comparing the estimated urine output at the goal time to the target urine output; and
generating, based on the comparing of the target urine output and the estimated urine output predicted by the machine learning model, an alert regarding whether the target urine output will be reached by the goal time.

18. The apparatus of claim 17, wherein the receiving further comprises receiving a plurality of input sets corresponding to a plurality of urine outputs and time values until goal time when the target urine output should be reached.

19. The apparatus of claim 18, wherein the predicting the estimated urine output is performed after a threshold quantity of input sets are received by the machine learning model.

20. The apparatus of claim 17, wherein the measurement of urine output indicates a volume of urine or a weight of urine, wherein the urine is collected via a Foley catheter over an interval of time.

21. The apparatus of claim 17, wherein the input set comprises a total amount of urine output at a measurement time, an average of an hourly urine output at the measurement time, a standard deviation of the hourly urine output at the measurement time, an average hourly urine output during a second half of a urine monitoring session, a previous hour's urine output, an elapsed time since a start of the urine monitoring session, and/or a time remaining until the goal time.

22. The apparatus of claim 17, wherein the goal time and the target urine output are configured via a user interface and provided to the apparatus.

23. The apparatus of claim 17, wherein the machine learning model comprises gradient boosting regression trees machine learning model.

24. The apparatus of claim 17, wherein the machine learning model is trained, and/or wherein the training is based on at least non-parametric statistical learning.

25. The apparatus of claim 17, wherein the machine learning model comprises a linear regression machine learning model, a random forest machine learning model, a neural network machine learning model, a gradient boosting regressor machine learning model, and/or a Kalman filter.

26. The apparatus of claim 17 further comprising providing the alert to a display to present an indication of whether the target urine output will be reached by the goal time.

27. The apparatus of claim 17, wherein the alert is used to assess whether to change a dosage of a medicament affecting urine output.

28. The apparatus of claim 17, wherein the comparing is performed by a comparison logic and/or the machine learning model.

29. The apparatus of claim 17, wherein the machine learning model comprises an ensemble of machine learning models.

30. The apparatus of claim 17, wherein the ensemble of machine learning models comprises a first machine learning model that predicts a first quantile of a first estimated urine output at the goal time, a second machine learning model that predicts a second quantile of a second estimated urine output at the goal time, and a third machine learning model that predicts a third quantile of a third estimated urine output at the goal time, wherein the first estimated urine output, the second estimated urine output, and the third estimated urine output provide a confidence interval for the estimated urine output at the goal time.

31. The apparatus of claim 30, wherein the comparing compares the first estimated urine output, the second estimated urine output, and the third estimated urine output to the target urine output.

32. The apparatus of claim 17, wherein the machine learning model is comprised in a urine collection system, is coupled via a wired or a wireless connection to the urine collection system, is provided as a service to one or more urine collection systems.

33. A non-transitory computer-readable storage medium including code which when executed by at least one processor causes operations comprising:
receiving, at a machine learning model, an input set comprising a measurement of urine output and a time value indicative of time remaining from when the first measurement is performed until a goal time when a target urine output should be reached;
predicting, by the machine learning model and using at least the measurement and the time value, an estimated urine output at the goal time;
comparing the estimated urine output at the goal time to the target urine output; and
generating, based on the comparing of the target urine output and the estimated urine output predicted by the machine learning model, an alert regarding whether the target urine output will be reached by the goal time.

34. A apparatus comprising:
means for receiving, at a machine learning model, an input set comprising a measurement of urine output and a time value indicative of time remaining from when the first measurement is performed until a goal time when a target urine output should be reached;
means for predicting, by the machine learning model and using at least the measurement and the time value, an estimated urine output at the goal time;
means for comparing the estimated urine output at the goal time to the target urine output; and
means for generating, based on the comparing of the target urine output and the estimated urine output predicted by the machine learning model, an alert regarding whether the target urine output will be reached by the goal time.

35. The apparatus of claim 34 further comprising means for performing any of the functions recited in any of claims 2-17.
